(19) United States
(12) Patent Application Publication
(10) Pub. No.: US 2011/0098216 A1
(43) Pub. Date: Apr. 28, 2011

(54) THERAPEUTIC USES OF COLOSTRININ

(75) Inventors: Istvan Boldogh, League City, TX (US); Marian L. Kruzel, Pearland, TX (US)

(73) Assignee: REGEN THERAPEUTICS PLC, London (GB)

(21) Appl. No.: 12/909,418

(22) Filed: Oct. 21, 2010

Related U.S. Application Data
(60) Provisional application No. 61/253,961, filed on Oct. 22, 2009.

(51) Int. Cl. A61K 38/02; A61P 3/04; A61P 3/10

(52) U.S. Cl. 514/4.8; 514/6.9

(57) ABSTRACT

There is provided the use of Colostrinin for the treatment of obesity.

### BACKGROUND OF THE INVENTION

(1) Field of the Invention

The present invention relates to Colostrinin and its use in therapy.

(2) Description of Related Art

More than 65 percent of adults in the United States are overweight or obese. Obesity puts people at increased risk for chronic diseases, such as cardiovascular diseases, endocrine disorders, type II diabetes, high blood pressure, psychological disorders, respiratory disorders, stroke, infertility, osteoarthritis and several forms of cancer. Recent studies have associated a systemic inflammatory response characterized by endothelial cell dysfunction, oxidative stress, and circulating immune cell activation with obesity. For instance, adipocytes release a variety of cytokines, such as IL-1 and TNF-alpha, and cytokine-like substances, such as leptin and resistin, which appear to mediate this inflammatory response. The inflammatory response may be exacerbated by the insulin resistance that is often associated with obesity. Obesity also puts people at increased risk to several other diseases such as asthma, breast cancer and non-alcoholic liver steatosis. Thus elucidating the connections between obesity and these various diseases is crucial for understanding the role and function of adipose tissue and adipocytokines on the body, and in particular, the cardiovascular system, and may contribute towards designing of future therapeutic approaches. Adipocytokines such as leptin, adiponectin, resistin and visfatin are bioactive mediators, and have been implicated in the regulation of metabolism, energy storage and homeostasis. Adipocytokines are released from cells such as adipocytes present in adipose tissue, as well as other cells such as epithelial and the various lymphatic and inflammatory cells present within fat tissue. These bioactive mediators play a major role in the pathogenesis of a cluster of clinical symptoms such as insulin resistance, obesity, atherosclerosis, dyslipidemia and hypertension. Further research has shown that obesity may aggravate microvascular dysfunction associated with pathological states, such as sepsis.

Thus there is a need for new and effective treatments for preventing and/or treating obesity.

Colostrum, or foremilk, is a viscous mammary gland secretion characterised by the presence of many elements needed by newborn mammals to develop properly. It is the first lacteal secretion post parturition and contains a high concentration of immunoglobulins (IgG, IgM and IgA) and non-specific proteins. It is replaced by mature breast milk about four to five days after birth.

Compared with mature breast milk, colostrum has low sugar content, but is richer in lipids, proteins, mineral salts and immunoglobulins. It also contains various floating cells such as granular and stromal cells, neutrophils, monocyte/macrophages and lymphocytes. It is also rich in growth factors, hormones and cytokines.

It has long been recognized that breast-feeding offers pronounced enhancement of passive immunity and promotes infantile gut immunity. Thus, the constituents of colostrum, and to a lesser extent also the mature milk, not only ensure adequate resistance to pathogens by delivery of maternal immunoglobulins and other protective factors, but also play a crucial role in promoting maturation of, inter alia, the immune and central nervous system.

Amongst the proteins present in colostrum, caseins are the most prevalent and known to form aggregates (micelles), which are similar in all mammals. Many proteins and peptides are bound to those aggregates, by weak hydrophobic and ionic forces. The resultant network of proteinaceous micelles has the ability to trap many small molecular weight compounds of differing nature, such as lipids, carbohydrates, and peptides, forming a unique homogeneous solution. The micelles help to distribute these micro-molecules relatively uniformly throughout the colostrum, and also prevent them from the formation of unwanted aggregates.

A number of peptides from milk with various biological activities have been reported. Some peptides exist naturally and some can be released via enzymatic proteolysis of the parent milk proteins. Besides casein protein, calcium and phosphate, the micelle also contains citrate, minor ions, lipase and plasmin enzymes, and various peptides entrapped in their structure.

Colostrinin, also known as colostrinine, proline-rich polypeptide or PRP, was first isolated in 1974 (Janusz et al, FEBS Lett., 49, 276-279) from ovine colostrum.

Certain therapeutic uses of colostrinin, particularly in the treatment of Alzheimer's disease, were described in WO98/14473, the contents of which are incorporated herein by reference. In this patent application, the physical characteristics of colostrinin, as determinable at the time, were described.

Although the physical characteristics were correct, the understanding of colostrinin has moved on since this application was filed. WO98/14473 also described a method for extracting colostrinin from raw colostrum. However this method has the disadvantages that the industrial scale up is difficult to obtain and yields from the method are low.

WO00/75173, the contents of which are incorporated herein by reference, describes a number of peptides found in colostrinin. WO02/46211, the contents of which are incorporated herein by reference, describes a number of other peptides which can be found in colostrinin.

Of considerable interest in colostrinin is the presence of various polypeptides that are more abundant in colostrum, than mature milk. During the days following parturition, the concentration of colostrinin in a mammary gland secretion precipitously diminishes through the end of the third day after delivery. Such a short lifetime for some of the colostrinin peptides indicates their important role in early development of infant's immune system and the protection of newborns against environmental shocks.

The colostrinin complex is now believed to consist of at least five subgroups of peptides; each subgroup has its own characteristic hydrophobic pattern. Evidence suggests that these peptides have a tendency to form aggregates, due to the presence of specially arranged non-polar, polar, aromatic, positively- and negatively-charged amino acids. Furthermore, the amino acid compositions of the peptides and their hydrophobic character further suggest this aggregating ability. It is believed that colostrinin is a mixture of more than 100 separate peptides, derived from precursor proteins, such as annexin, beta-casein, a hypothetical beta-casein homologue and others, including those with no homology to any specific protein in the current GenBank database.

### BRIEF SUMMARY OF THE INVENTION

We have now found that Colostrinin has a number of previously unknown therapeutic effects. More particularly, we have found that Colostrinin is useful in the prevention and/or treatment of obesity and related disorders.

The present invention provides a use of, and a method of treatment involving, Colostrinin, which has been found to have applications in the prevention and/or treatment of obesity and/or obesity-related disorders.

### BRIEF DESCRIPTION OF THE DRAWING FIGURES

FIG. 1 is a graph showing the lifespan in days of mice fed with Colostrinin™/proline-rich polypeptide (“CLN”) as compared to mice fed with control substances.

FIG. 2 includes three graphs showing the effect of CLN on learning and memory in young, middle-aged and old animals, respectively, as compared to controls.

FIG. 3 is a graph showing the effect of CLN on the body weight of senescence-prone mice (SAMP) as compared to colostrum administration and BSA hydrolysate administration.

### DETAILED DESCRIPTION OF THE INVENTION

According to one aspect of the invention there is provided the use of Colostrinin in the manufacture of a medicament for treating and/or preventing obesity and/or disorders related to obesity. Colostrinin may be used as a medicament for non-rodent mammals; we have found that Colostrinin is especially useful as a medicament for the treatment of humans.

According to another aspect of the invention there is provided Colostrinin for use in the treatment and/or prevention of obesity and/or disorders related to obesity.

According to another aspect of the invention there is provided Colostrinin for use in the control of body weight.

In addition to the foregoing, Colostrinin may be used to prevent and/or treat obesity-related disorders, including type II diabetes mellitus; hyperlipidemia; dyslipidemia; abdominal obesity; hypercholesterolemia; hypertrigyceridemia; atherosclerosis; coronary heart disease; stroke; hypertension; peripheral vascular disease; vascular restenosis; nephropathy; neuropathy; inflammatory conditions, such as, but not limited to, irritable bowel syndrome, inflammatory bowel disease, including Crohn's disease and ulcerative colitis; other inflammatory conditions; pancreatitis; neurodegenerative disease; retinopathy; neoplastic conditions, such as, but not limited to adipose cell tumors, adipose cell carcinomas, such as liposarcoma; cancers, including gastric and bladder cancers; angiogenesis; Alzheimer's disease; psoriasis; and other disorders where insulin resistance is a component. Colostrinin may also be useful in the treatment, control and/or prevention of overeating; bulimia; elevated plasma insulin concentrations; insulin resistance; glucose tolerance; Metabolic Syndrome; lipid disorders; low HDL levels; diabetes while mitigating cardiac hypertrophy, including left ventricular hypertrophy; high LDL levels; hyperglycemia; neoplastic conditions, such as endometrial, breast, prostate, kidney and colon cancer; osteoarthritis; obstructive sleep apnea; gallstones; abnormal heart rhythms; heart arrythmias; myocardial infarction; congestive heart failure; sudden death; ovarian hyperandrogenism, (polycystic ovary disease); craniopharyngioma; the Prader-Willi Syndrome; Frohlich's syndrome; GH-deficient subjects; normal variant short stature; Turner's syndrome; and other pathological conditions showing reduced metabolic activity or a decrease in resting energy expenditure as a percentage of total fat-free mass, e.g., children with acute lymphoblastic leukemia.

In addition to the foregoing, Colostrinin has been found to alter the gene expression of resistin. Thus, in accordance with the present invention, there is provided the use of Colostrinin to modulate the gene expression of resistin in a cell.

Resistin, also known as Serine/Cysteine-rich Adipocyte-Specific Secretory Factor, is a hormone secreted by adipose tissue. When first discovered, the term resistin was coined due to the observed resistance to insulin in mice injected with this hormone. Resistin comprises a dimer of two 92 amino acid polypeptides; the pre-peptide form of resistin in human is 108 amino acids in length, with a molecular weight of about 12.5 kiloDaltons. It is one of a variety of hormones synthesized and released from adipose tissue, including adiponectin, angiotensin, estradiol, IL-6, leptin, PAI-1 and TNF-α. Resistin is thought to serve endocrine functions likely involved in insulin resistance. Further research has suggested a role for resistin to other physiological systems, for instance, obesity and energy homeostasis.

Resistin has also been implicated in the induction of inflammation. Leukocyte recruitment (for instance neutrophils and mast cells) at sites of infection or irritation due to the innate immune response results in leukocyte accumulation and secretion of inflammatory agents such as histamine, prostaglandin and pro-inflammatory cytokines; resistin has been shown to be associated in these inflammatory responses. For instance, resistin has been shown to increase gene expression of several pro-inflammatory cytokines, such as interleukin-1 (IL-1), interleukin-6 (IL-6), interleukin-12 (IL-12), and tumour necrosis factor-α (TNF-α) in an NFκB-mediated fashion. Furthermore studies have shown that resistin upregulates expression of intracellular adhesion molecule-1 (ICAM1), vascular cell-adhesion molecule-1 (VCAM1) and CCL2, all of which are involved in leukocyte recruitment to sites of infection or irritation. As resistin has been implicated in insulin resistance, resistin may well be involved in the well-known association between inflammation and insulin resistance.

Recent studies have shown a strong relationship between obesity, insulin resistance, and chronic inflammation, thereby suggesting that resistin (and/or its associated signalling pathways), at least in part, may serve as a link between obesity and type II diabetes mellitus, in addition to contributing to the inflammatory response. Nevertheless, resistin certainly bears features of a pro-inflammatory cytokine, thus resistin may well have a role in inflammatory diseases regardless of resistin's putative role in insulin resistance.

The strong relationship observed between serum resistin levels and obesity has suggested a role for resistin in energy metabolism and type II diabetes mellitus; for instance, increased serum resistin levels have been observed with increased adiposity. Other studies have demonstrated positive correlations between resistin levels and insulin resistance, as well as direct correlations between resistin levels and subjects with type II diabetes mellitus. Thus some researchers have suggested the link between increased resistin serum levels and the insulin resistance apparently associated with increased adiposity.

Thus, the downregulation of resistin may reduce the innate immune response of a cell to an infection or irritation. Alternatively, the downregulation of resistin may downregulate the expression of pro-inflammatory cytokines such as IL-1, IL-6, IL-12 and TNF-α, and downregulate the expression of ICAM1, VCAM1 and CCL2. Thus, advantageously, in accordance with the present invention Colostrinin may be used to prevent and/or treat inflammatory disorders.

Furthermore, the downregulation of resistin may prevent and/or treat type II diabetes mellitus. Thus, advantageously, in accordance with the present invention Colostrinin may be used to prevent and/or treat type II diabetes mellitus.

Furthermore, the downregulation of resistin may prevent and/or treat obesity. Thus, advantageously, in accordance with the present invention Colostrinin may be used to prevent and/or treat obesity.

In addition to the foregoing, Colostrinin may be used to prevent and/or treat other inflammatory disorders. An inflammatory disorder may be either an acute or chronic inflammatory disorder, which can result from infections or non-infectious causes. Infectious conditions include meningitis, encephalitis, uveitis, colitis, dermatitis, and adult respiratory distress syndrome. Non-infectious causes include trauma (burns, cuts, contusions, crush injuries), autoimmune diseases, and organ rejection episodes. Thus an inflammatory disorder may be a condition selected from a group comprising: atherosclerosis (arteriosclerosis); autoimmune conditions, such as multiple sclerosis, systemic lupus erythematosus, polymyalgia rheumatica (PMR), rheumatoid arthritis and other forms of inflammatory arthritis, Sjogren's Syndrome, progressive systemic sclerosis (scleroderma), ankylosing spondylitis, polymyositis, dermatomyositis, pemphigus, pemphigoid, Type I diabetes mellitus, myasthenia gravis, Hashimoto's thyroditis, Graves' disease, Goodpasture's disease, mixed connective tissue disease, sclerosing cholangitis, inflammatory bowel disease including Crohn's Disease (regional enteritis) and ulcerative colitis, pernicious anemia, inflammatory dermatoses; usual interstitial pneumonitis (UIP), asbestosis, silicosis, berylliosis, talcosis, all forms of pneumoconiosis, sarcoidosis (in the lung and in any other organ), desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa); inflammatory dermatoses not presumed to be autoimmune; chronic active hepatitis; delayed-type hypersensitivity reactions (e.g., poison ivy dermatitis); pneumonia or other respiratory tract inflammation due to any cause; Adult Respiratory Distress Syndrome (ARDS) from any etiology; encephalitis, with inflammatory edema; immediate hypersensitivity reactions including, but not limited to, asthma, hayfever, cutaneous allergies, acute anaphylaxis; diseases involving acute deposition of immune complexes, including, but not limited to, rheumatic fever, acute and/or chronic glomerulonephritis due to any etiology, including specifically post-infectious (e.g., post-Streptococcal) glomerulonephritis, acute exacerbations of Systemic Lupus Erythematosus; pyelonephritis; cellulitis; cystitis; acute cholecystitis; and conditions producing transient ischemia anywhere along the gastrointestinal tract, bladder, heart, or other organ, especially those prone to rupture; sequalae of organ transplantation or tissue allograft, including allograft rejection in the acute time period following allogeneic organ or tissue transplantation and chronic host-versus-graft rejection.

In addition to the foregoing, Colostrinin has been found to alter the gene expression of leptin. Thus, in accordance with the present invention, there is provided the use of Colostrinin to modulate the gene expression of leptin in a cell.

Leptin is a 167 amino-acid peptide, encoded by the ob gene. Adipocytes are the primary sites of leptin production, but it was also shown to be generated by gastric epithelial cells, endothelial cells, placenta, ovary, skeletal muscle and liver. Leptin provides a signal through its actions on CNS receptors within the hypothalamus. Mice with mutated ob gene (ob/ob mice) develop obesity in relation to the lack of satiety signalling within their brain gut axis. Adult animals with leptin deficiency show increased appetite and obesity which can be treated by leptin. These animals also exhibit T cell hypo-responsiveness, hyperinsulinemia and insulin resistance, hyperlipidemia, immune dysfunction, and neuroendocrine abnormalities.

Plasma levels of leptin in humans are closely associated with the fat mass. Like the majority of neurohormones, leptin levels exhibit important circadian rhythms. Several agonists including TNF-alpha and other pro-inflammatory cytokines, insulin, glucose, and estrogens have been shown to increase leptin release from adipocytes. Increased levels of other vasoactive factors like angiotensin II or endothelin may also lead to leptin generation, although this phenomenon may occur locally since it does not seem to affect plasma levels of leptin during angiotensin II administration. Leptin receptors are widely expressed on various cells including cells of the cardiovascular and immune system.

Furthermore, the modulation of leptin gene expression may prevent and/or treat obesity. Furthermore the modulation of leptin gene expression and the modulation of resistin gene expression may synergistically prevent and/or treat obesity. Thus, advantageously, in accordance with the present invention Colostrinin may be used to prevent and/or treat obesity.

In general, a chronic disorder is a disorder that has persisted for a long time, usually at least one week, more usually at least one month, and often at least 3 months or at least 6 months.

It is a feature of the present invention to use Colostrinin for improving the development of body mass of a new born child. It is a further feature of the invention to use Colostrinin to correct deficiencies of body mass in a child. These uses of the Colostrinin may be particularly applicable to babies or children who have been deprived of colostrum (formula fed babies). This may occur, for example, in babies and children who were not breast fed from birth; the artificial feed that such babies and children would have been given does not contain Colostrinin.

According to another aspect of the invention, we provide the use of Colostrinin as a dietary supplement for the treatment and/or prevention of obesity and/or obesity-related disorders. Colostrinin may advantageously be used as a dietary supplement for middle-aged and old adults who are obese, or adults who have been predicted to become obese later in life. Alternatively, Colostrinin may be used as a dietary supplement for babies and young children to correct deficiencies in the development of their body weight. As noted above, such deficiencies may arise in babies and children who had not been breast fed from birth. In an aspect of the invention, we provide a dietary supplement comprising an orally ingestible combination of Colostrinin in combination with a physiologically acceptable carrier. The dietary supplement may be provided in liquid or solid form; the dietary supplement may suitably be provided in the form of a tablet.

In accordance with the invention, Colostrinin may be administered prophylactically in order to help to prevent the development of obesity and/or disorders related to obesity.

The Colostrinin used in the present invention may be ovine Colostrinin, or it may be non-ovine Colostrinin. Non-ovine Colostrinin may be derived from the colostrum of, for example, humans, cows, horses, buffaloes, goats, pigs, yaks, llamas and asses. The colostrum will normally be present in the beestings of these animals for 1 to 4 days after parturition.

The term “Colostrinin”, as used herein refers to a complex of small molecular weight milk-derived proline-rich polypeptides which, in its natural form, is obtained from mammalian colostrum.

The amino acid composition of ovine Colostrinin comprises approximately 22% by weight proline and a high proportion of non-polar amino acids (hydrophobic amino acids constitute approximately 50% of the total protein). Colostrinin is not phosphorylated or glycosylated. The amino acid composition of Colostrinin is very similar in all mammalian species. However, due to individual variations of colostrum it is very difficult to obtain identical end-product.

Colostrinin may be obtained by a number of methods. WO2004/081038, the contents of which are incorporated herein by reference, describes a method, comprising alcohol extraction and membrane filtration steps, to obtain colostrum-derived Colostrinin. According to this method, avoidance of excessively harsh conditions during the purification procedure preserves the peptides' structure and biological activity. Colostrinin obtained by using this method appears to be consistent in molecular size distribution of the peptides according to SDS-PAGE and high performance liquid chromatography (HPLC); similarity in amino acid composition, characterized by high content of proline; and ability to induce cytokines (e.g. IFN gamma).

According to another method developed by Janusz et al., Colostrinin may be obtained from colostrum by pH dependent casein precipitation followed by various chromatographic steps, including ion exchange, affinity and molecular sieving, combined with ammonium sulphate precipitation. Although this method is reproducible, it is laborious and difficult to scale up for industrial applications.

Whilst the above definition relates to naturally occurring mammalian Colostrinin, the term Colostrinin as herein used also includes analogues thereof having substantially the same biological activity produced by recombinant DNA technology. Colostrinin as used herein also includes complexes of biologically active polypeptides of substantially the same composition as natural Colostrinin, which have been made by polypeptide synthesis.

In a further aspect of the present invention there is provided a method of treating and/or preventing obesity and/or disorders related to obesity. The disorders that can, with advantage, be treated using the method according to the invention are described above. In a preferred embodiment the Colostrinin is administered to a patient daily at 1 to 2 therapeutic units. In another embodiment the Colostrinin is administered to a patient for a first period at about 1 to 2 therapeutic units daily, followed by a second period when no Colostrinin is administered. The first period is preferably about 2 to 4 weeks, more preferably about 3 weeks; and the second period is preferably about 2 to 5 weeks, more preferably about 4 weeks. This cycle is preferably repeated at least once, and is more preferably repeated more than once.

The therapeutic unit for use in methods of the invention is preferably in the range 10 to 10,000 micrograms of Colostrinin, more preferably in the range of 25 to 2000 micrograms, most preferably in the range of 50 to 500 micrograms.

The Colostrinin may be formulated for administration in any suitable form. For example, it may be formulated for oral, rectal or parenteral administration. More specifically, the Colostrinin may be formulated for administration by injection, or, preferably, in a form suitable for absorption through the mucosa of the oral/nasopharyngeal cavity, from the alimentary canal or any other mucosal surface. The oral formulations may be provided in a form for swallowing; or, preferably, in a form for dissolving in the saliva, whereby the formulation can be absorbed in the mucous membranes of the oral/nasopharyngeal cavity. The oral formulations may be in the form of a tablet for oral administration, lozenges (i.e. a sweet-like tablet in a form suitable to be retained in the mouth and sucked), adhesive gels for rubbing into the gum. The Colostrinin may be formulated as an adhesive plaster or patch, which may be applied to the gums. The Colostrinin may also be formulated for application to mucous-membranes of the genito-urinary organs.

Colostrinin for use in the present invention may be obtained from any mammal, including human sources or animals such as cows, horses, buffaloes, goats, pigs, yaks, sheep, llamas or asses, camels etc. Tests on Colostrinin were performed using ovine, bovine and human Colostrinin.

Further toxicological study of Colostrinin for use in the present invention showed:
a) no mutagenic potential by measuring its ability to induce reverse mutations at selected loci of several strains of Salmonella typhimurium and at the tryptophan locus of Escherichia coli strain WP2 uvrA in the presence and absence of Aroclor-induced rat liver S9,
b) no clastogenic potential based upon its ability to induce chromosome aberrations in CHO cells,
c) no clastogenic potential as measured by its ability to induce micronucleated polychromatic erythrocytes in mouse bone marrow.

In natural conditions analogues of ovine Colostrinin but possessing the biochemical properties thereof are present in human colostrum and in the beestings of all mammals, and especially large farm animals such as cows, horses, goats, pigs, yaks, buffaloes, sheep, llamas or asses, camels etc. for 1-4 days after parturition. The Colostrinin enters the body of the newborn animals during sucking and swallowing of its mother's colostrum. Owing to the low molecular weight it is possible for Colostrinin to act on the mucosa of the oral/nasopharyngeal cavity via cell receptors, and even as a result of ordinary diffusion. Thus Colostrinin can be administered in the form of tablets and sublingual tablets for sucking, tablets and capsules for swallowing, in the form of adhesive gels, and in the form of adhesive plasters for fastening to the gums. Colostrinin can also be applied to the mucous membranes of genito-urinary organs.

Scientific studies aiming to elucidate the biochemical activity induced by Colostrinin also revealed the existence of similar biological activity when using human colostrum, collected from women in the period of 1-7 days after parturition. The method used for isolating Colostrinin of human origin was analogous to the methods of obtaining it from the beestings of farm animals, and especially from sheep beestings. It was found that the colostrum secreted by the mammary gland of women for first week after parturition contains Colostrinin, the amount of which depends on lactation and is optimum between 2-3 days, when about 300 mg of it is detected in 1 litre of colostrum. This quantity of human Colostrinin is within the range seen in ovine colostrum. It was demonstrated that human Colostrinin has many biological properties similar to, for example, the sheep analogue.

These biological activities of the Colostrinin both of human and of animal origin were determined by the method known from a Polish patent (PL 170592B1) and from European patent no. EP-B-0609225, pt.: Testing Immunology.

In studies on human volunteers it was shown that after administration of tablets for sucking, containing at least 25 μg of Colostrinin at doses of at least one daily, for a period of about 3 weeks, no adverse reactions were observed.

In order that the invention may be more fully understood, reference will now be made to the accompanying Examples by way of illustration only.

### Example 1

Mutation Assay using Salmonella typhimurium tester strains TA98, TA100, TA1535 and TA1537 and Escherichia coli tester strain WP2 uvrA in the presence and absence of Aroclor-induced rat liver S9. The assay was performed in two phases, using the plate incorporation method. The first phase, the initial toxicity-mutation assay, was used to establish the dose-range for the confirmatory mutagenicity assay and to provide a preliminary mutagenicity evaluation. The second phase, the confirmatory mutagenicity assay, was used to evaluate and confirm the mutagenic potential of the test article, Colostrinin™/proline-rich polypeptide, hereafter referred to as Colostrinin™. The dosing solutions, except those from the parallel lactose experiment, were adjusted to compensate for the protein content of the freeze-dried material (13.6% protein) using a correction factor of 7.353. Water was selected as the solvent of choice based on the ability of the test article to form a workable suspension in water, the Sponsor's request and compatibility with the target cells. The test article formed workable suspensions in water from approximately 50 to 200 mg/mL after sonication at 29° C. for 15 minutes.

In the initial toxicity-mutation assay, the maximum dose tested was 5000 μg per plate; this dose was achieved using a concentration of 10 mg/mL and a 500 μL plating aliquot. The dose levels tested were 1.5, 5.0, 15, 50, 150, 500, 1500 and 5000 μg per plate. The test article formed a workable suspension in water at 10 mg/mL, soluble but cloudy solutions from 0.030 to 3.0 mg/mL and soluble and clear solutions from 0.0030 to 0.010 mg/mL. In the initial toxicity-mutation assay, no positive mutagenic response was observed. Precipitate was observed beginning at 1500 or at 5000 μg per plate. No appreciable toxicity was observed.

Based on the findings of the initial toxicity-mutation assay, the maximum dose plated in the confirmatory mutagenicity assay was 5000 μg per plate. The test article contained 13.6% protein and the remainder of the material (86.4%) was lactose.

In order to determine the relative toxicity of the lactose portion of the test article to the protein portion, a sample of lactose was also tested. In the initial toxicity-mutation assay with lactose, the maximum dose tested was 31765 μg per plate; this dose was achieved using a concentration of 63.53 mg/mL and a 500 μL plating aliquot. The dose levels tested were 0.019, 0.064, 0.19, 0.64, 1.9, 6.4, 19 and 63.53 mg/mL. Lactose formed a soluble but cloudy solution in water at approximately 63.53 mg/mL and soluble and clear solutions from approximately 0.19 to 19 mg/mL. Neither precipitate nor appreciable toxicity was observed; therefore, a confirmatory assay was not plated using lactose. In the confirmatory mutagenicity assay, no positive mutagenic response was observed. The dose levels tested were 50, 150, 500, 1500 and 5000 μg per plate. Precipitate was observed at 5000 μg per plate. No appreciable toxicity was observed.

Under the conditions of this study, test article Colostrinin™ proline-rich polypeptide was concluded to be negative in the Bacterial Reverse Mutation Assay.

### Example 2

The test article, Colostrinin™/proline-rich polypeptide, hereafter referred to as Colostrinin™, was tested in the chromosome aberration assay using Chinese hamster ovary (CHO) cells in both the absence and presence of an Aroclor-induced S9 activation system. A preliminary toxicity test was performed to establish the dose range for the chromosome aberration assay. The chromosome aberration assay was used to evaluate the clastogenic potential of the test article. The dosing solution concentrations were adjusted to compensate for the protein content (13.6%) of the test article using a correction factor of 7.35.

In order to evaluate the toxic effect of lactose alone on the test system, α-Lactose monohydrate was tested as reference article at the same percentage of lactose as in the highest test article dose formulation in the preliminary toxicity assay. McCoy's media (complete medium for the non-activated test system and serum-free medium for the S9-activated test system) were the solvents of choice based on the solubility of the test article and compatibility with the target cells. The test article formed a workable suspension in McCoy's complete medium at a maximum concentration of approximately 200 mg/mL in the solubility test. The test article formed a workable suspension in McCoy's serum-free medium at a concentration of 36.76 mg/mL in the preliminary toxicity assay. The reference article was soluble in McCoy's complete and serum-free media at the dose level tested in the preliminary toxicity assay.

In the preliminary toxicity assay, the maximum dose tested was 5000 μg/mL. The test article formed a workable suspension in complete medium at concentrations ≧3.676 mg/mL and concentrations ≦1.103 mg/mL were soluble in complete medium. The test article formed a workable suspension in serum-free medium at concentrations ≧1.103 mg/mL and concentrations ≦0.367 mg/mL were soluble in serum-free medium. Visible precipitate was observed in treatment medium at dose levels ≧500 μg/mL and dose levels ≦150 μg/mL were soluble in treatment medium at the beginning and conclusion of the treatment period. The reference article was soluble in complete medium and serum-free medium at 31.76 mg/mL at the beginning and conclusion of the treatment period.

Substantial test article toxicity (i.e., at least 50% cell growth inhibition, relative to the solvent control) was not observed in either the test article or the reference article at any dose level in all three exposure groups. Based on these findings, the doses chosen for the chromosome aberration assay ranged from 62.5 to 500 μg/mL for all three exposure groups. Since substantial toxicity was not observed in the reference article, it was not tested in the chromosome aberration assay.

In the chromosome aberration assay, the cells were treated for 4 and 20 hours in the non-activated test system and for 4 hours in the S9-activated test system. All cells were harvested 20 hours after treatment initiation. The test article formed a workable suspension in complete medium at 3.676 mg/mL and concentrations ≦1.838 mg/mL were soluble in complete medium. The test article formed a workable suspension in serum-free medium at concentrations ≧1.838 mg/mL and concentrations ≦0.919 mg/mL were soluble in serum-free medium. Visible precipitate was observed in complete medium at 500 μg/mL and dose levels <250 μg/mL were soluble in complete medium at the beginning and conclusion of the treatment period. Visible precipitate was observed in serum-free medium at dose levels ≧250 μg/mL and dose levels <125 μg/mL were soluble in serum-free medium at the beginning of the treatment period. At the conclusion of the treatment period, visible precipitate was observed in serum-free medium at 500 μg/mL and dose levels ≦250 μg/mL were soluble in serum-free medium. In the absence of substantial toxicity at any dose level, selection of doses for microscopic analysis was based on precipitation (the highest dose with visible precipitation and two lower doses) in all harvests. The percentage of cells with structural or numerical aberrations in the test article-treated groups was not statistically increased above that of the solvent control at any dose level (p>0.05, Fisher's Exact test).

Based on the findings of this study, Colostrinin™ was concluded to be negative for the induction of structural and numerical chromosome aberrations in CHO cells in both the non-activated and the S9-activated test systems.

### Example 3

The test article, Colostrinin™/proline-rich polypeptide, hereafter referred to as Colostrinin™, was tested in the mouse micronucleus assay. The assay was performed in two phases. The first phase, pilot toxicity study, was designed to assess toxicity of the test article and set dose levels for the definitive study. The second phase, the definitive micronucleus study, was designed to evaluate the potential of the test article to increase the incidence of micronucleated polychromatic erythrocytes in bone marrow of male and female ICR mice.

Phosphate buffered saline (PBS) was used as the test article vehicle. The test article was a workable suspension in PBS at 100 mg/mL, the maximum concentration prepared and tested in this study. All test article dose concentrations were corrected for the 13.6% protein content of the test article (freeze-dried material) using a correction factor of 7.35. In the definitive phase of the study, PBS was used as the vehicle (negative) control article and cyclophosphamide monohydrate (CP), at a dose of 50 mg/kg, as the positive control article. In both phases of the study, test or control articles were administered at a dose volume of 20 mL/kg body weight via a single oral gavage dose. In the pilot toxicity study, two male mice each were exposed to Colostrinin™ proline-rich polypeptide, at a dose of 1, 10, 100 or 1000 mg/kg body weight and five male and five female mice were exposed to 2000 mg/kg. In addition, five male and five female mice were dosed with 86.4% α-lactose monohydrate (lactose) formulated in PBS in order to determine the animal tolerance on the high percent of lactose present in the test article. No mortality or clinical signs were observed during the course of the study. All animals treated with the test article or 86.4% lactose were normal in appearance and behavior. Since animals dosed with the lactose exhibited no clinical signs, the effect of lactose was not further investigated. In the absence of mortality, the high dose for the micronucleus test was set at 2000 mg/kg.

The definitive micronucleus study consisted of seven groups, each containing 5 male and female ICR mice. Mice in five of these groups were treated either with the controls (vehicle or positive) or with Colostrinin™ proline-rich polypeptide at a dose of 500, 1000 or 2000 mg/kg and were euthanized 24 hours after treatment. Mice in the other two groups were treated either with the vehicle control article or Colostrinin™ proline-rich polypeptide at a dose of 2000 mg/kg and were euthanized 48 hours after treatment. An additional group of 5 male and 5 female ICR mice were treated with 2000 mg/kg to be used as possible replacement animals for the high dose in the event of mortality. No mortality or clinical signs were observed in any male or female mice in any of the treatment groups. All animals were normal in appearance and behavior during the course of the study. Bone marrow cells [polychromatic erythrocytes (2000 PCEs/animal)] collected 24 and 48 hours after treatment were examined microscopically for the presence of micronuclei. Based on bone marrow analysis the following was observed:
No appreciable reduction in the ratio of polychromatic erythrocytes to total erythrocytes in the test article groups relative to the vehicle control groups were observed suggesting that the test article did not inhibit erythropoiesis.
No significant increase in the incidence of micronucleated polychromatic erythrocytes in the test article-treated groups relative to the respective vehicle control groups was observed in male or female mice at 24 or 48 hours after dose administration (p>0.05, Kastenbaum-Bowman Tables).
CP, the positive control, induced a significant increase in the incidence of micronucleated PCEs (p≦0.05, Kastenbaum-Bowman Tables) in both male and female mice. The number of micronucleated PCEs in the vehicle control groups did not exceed the historical vehicle control range.

These results indicate that all criteria for a valid test were met as specified in the protocol and that there was no problem with the test system or the quality of the test. The results of the assay indicate that under the conditions described in this report, a single oral administration of Colostrinin™ proline-rich polypeptide at doses up to and including 2000 mg/kg did not induce a significant increase in the incidence of micronucleated polychromatic erythrocytes in bone marrow of male and female ICR mice. Therefore, Colostrinin™ proline-rich polypeptide was concluded to be negative in the mouse micronucleus assay.

### Example 4

Senescence-prone mice, or SAMP, (Harlan Sprague Dawley, Inc., Madison, Wis., USA) were kept in clear polycarbonate cages (six mice per unit), maintained at 23 ±1° C., under a 12 h light/dark cycle. Mice had free access to food and water except during the testing sessions. Mice were fed with the standard NIH-31 formulation. Test samples received Colostrinin™/proline-rich polypeptide (ReGen Therapeutics, Plc. London, England), hereafter referred to as CLN, and controls received standard bovine colostrum, phosphate buffered saline (PBS; solvent), or bovine serum albumin hydrolysate (BSA hydr.), via drinking water. The concentrations were set so that individual mice obtained 1 μg and 0.1 μg CLN per kg per day.

The genetic factors involved in the body weight, senescence processes and lifespan in SAMP mice are highly complex. For example, the genes responsible for the accelerated senescence appear to be recessive genetically. We measured lifespan, body weight, as well as spatial learning and memory in CLN-fed and control animals.

Unless otherwise indicated, mice were fed with CLN from 3 month of age for their entire lifespan. Control mice received same amounts of colostrum, BSA hydr. or the solvent (PBS). CLN-fed group: male mice (n=40) and female mice (n=45) were used. Colostrum-fed group: male mice (n=41), female mice (n=40), solvent-fed group: male mice (n=37), female mice (n =34). A group of animals (female: n=27; male n=31) received no supplementation (No-treatment).

The lifespan of the control mice (colostrum or PBS-treated) ranged from 293 to 651 days with a mean value±standard deviation of 466.3±121.2 days. The mean lifespan of males and females were 463.8±115.2, and 482±111.2 days, respectively. There were no significant differences in the mean lifespan between female and male mice.

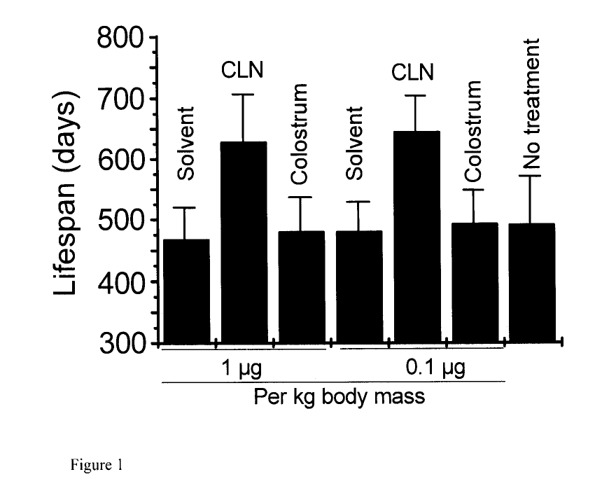

As compared to controls, CLN-fed animals showed a remarkable extension of the mean lifespan, as shown in FIG. 1. Overall there was a 34.9% increase in mean survival was compared to the controls. The lifespan of CLN-fed animals ranged from 572.5 days to 752.7 days. The mean lifespan of CLN-fed mice was 632.9±98.2 days. There are no significant differences between male and female groups. It should be noted that there are no notable differences in lifespan between groups fed with 1 μg and 0.1 μg per kg CLN.

Learning and memory were measured using a swim maze test to measure the ability of the mice to learn and remember the location of a hidden platform. This task is dependent on cortical and hippocampal functions. Briefly, in a given trial, the mice were allowed to swim in a steel tank, filled with opaque water maintained at 23 ±1° C. A hidden escape platform was provided below (1.5 cm) the surface of the water. The length of the path taken by the mice to reach the platform was recorded. During the pre-training period, mice were trained to swim and climb onto the platform, without learning its location in the tank. Subsequently, mice were tested for their ability to learn the location of the platform during two phases: acquisition (8 sessions), retention (2 sessions after a 2-day rest). During each session the mice had to reach the hidden platform from a various starting points in the tank. The length of the path taken to reach the hidden platform was analyzed to assess the efficiency with which the mice located the platform (independently of their speed of swimming). The data from most of the measures were subjected to two-way analyses of variance, with age and supplementation as between-groups factors. Planned individual comparisons of CLN-fed vs. control groups (colostrum, solvent) and between age-matched treatment groups were made using single-degree-of-freedom F tests.

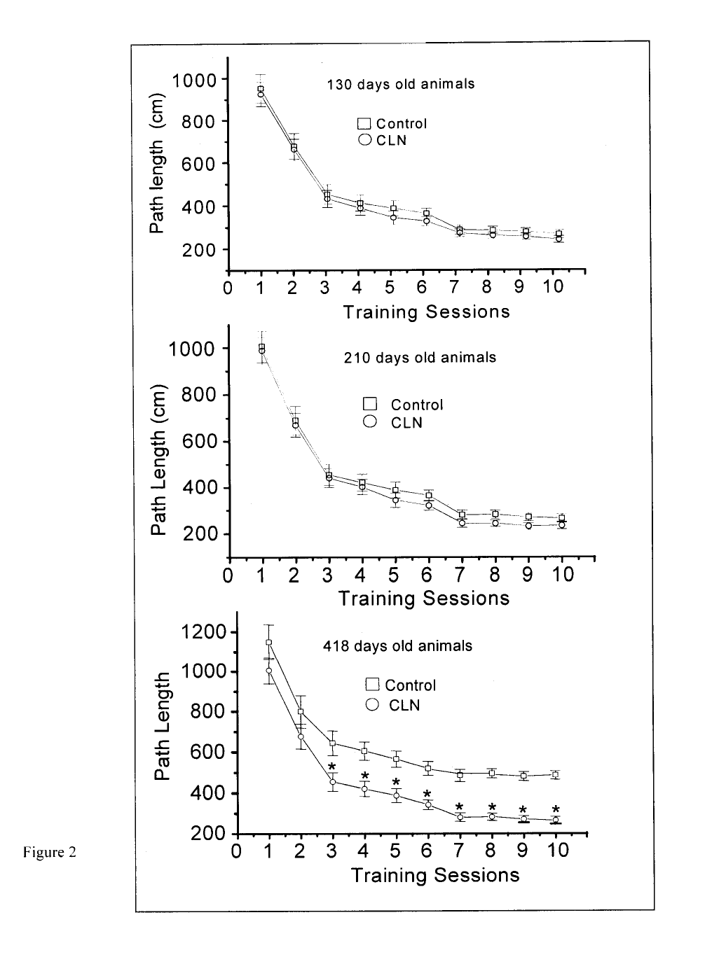

The effect of CLN on learning and memory in young, middle-aged and old animals is shown in FIG. 2. Young (130 days old) and middle-aged (210 days old), as well as old (418 days old), mice learned to locate the platform by the 6th session of the learning phase and maintained this level of performance during rest of our studies. The young and middle-aged mice showed a more rapid and better performance than the aged mice on sessions 2-5 (FIG. 2, upper, middle and lower panels). CLN administration to young or middle-aged mice showed a substantial, but not significant, effect on performance. On the other hand, CLN-fed 418 days old mice showed a significant improvement during the acquisition or retention phases compared to control (solvent-fed) animals, as determined by an individual comparison within three-way ANOVA (FIG. 2, lower panel, asterisks). When CLN-feeding was initiated at age 210 or even 420 days of animals life, there was significant (or substantial) improvement observed in spatial learning and memory compared to controls (data not shown).

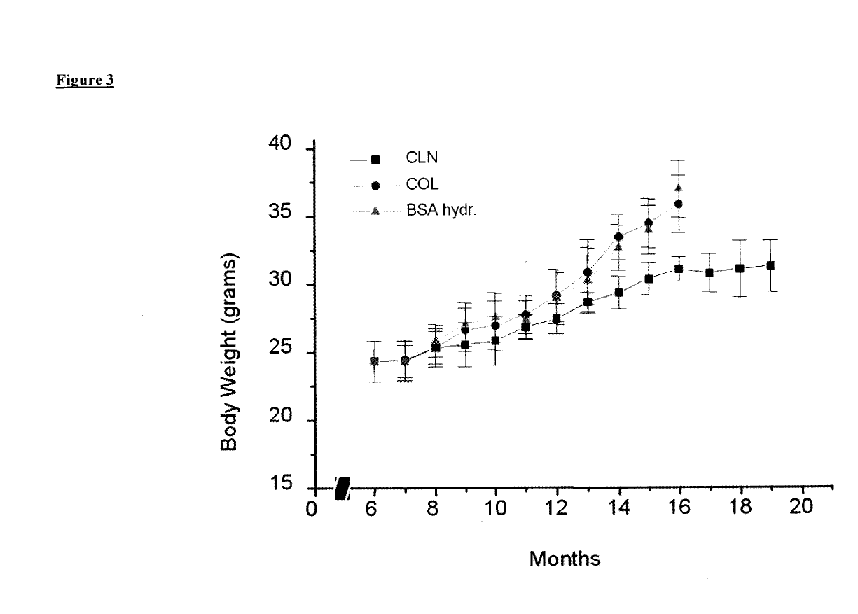

The effect of CLN on SAMP body weight was measured, compared to colostrum administration and BSA hydrolysate administration, as shown in FIG. 3. The body weights were measured monthly from 6 months of ages.

Colostrum conferred no benefit to SAMP over control (BSA hydrolysate), with regards to body weight or life span. Colostrinin administration resulted in lower body weight compared to the control group (BSA hydrolysate). The lower body weight of the Colostrinin-fed mice compared to the control group of the colostrum-fed group was more apparent at older ages (14-19 months); the Colostrinin-fed mice displayed approximately 10% lower body weights compared to colostrum-fed or the control mice at these ages.

Results of these studies show that CLN increases life-span, improves cognitive functions and is effective in preventing age-associated decline in cognitive functions. The results presented here indicate that CLN may have beneficial effects on brain functions therefore it may be used in preventive and therapeutic approaches in an attempt to decelerate aging processes and to improve symptoms of age-related neurodegenerative diseases in humans. The mechanistic basis (e.g., oxidative DNA damage, mitochondrial function) and beneficial effect of CLN on motor learning, maximum running performance, spontaneous locomotor activity, motor kills and sensory reactivity are the subject of future publications.

### Example 5

To gain insight into the molecular mechanisms by which CLN exerts its complex biological effects described in recent publications, we performed microarray analysis of CLN-induced changes in the gene expression profiles (GEPs) of CLN-treated cells. We used TR146 cells, an established model cell line for human digestive system epithelium. These cells were used because they are primarily exposed by orally administered CLN. Parallel cultures of cells were treated with 100 ng per ml CLN and RNAs were isolated at 3, 6, 12 and 18 hours and subjected to microarray analysis. We identified the differentially expressed genes at 3, 6, 12 and 18 hours after CLN treatment by the following criteria: 1) in each sample genes with normalized expression values ≧3.24×10−5 (corresponding to a signal ≧200 in the control sample) were considered present, 2) only genes that were found present in the control sample, or in the CLN-treated sample, (or in both), were subjected to fold-change analysis, 3) genes with at least 2-fold expression change compared to control were selected as differentially expressed. The numbers of differentially expressed genes were found to be 450, 27, 107, 318 at 3, 6, 12, and 18 hours respectively.

A novel finding of this study was that CLN treatment modulates the cells' mRNA production for resistin. For example, resistin expression was downregulated 5.01-fold. We generated a molecular network based on the differentially expressed genes in the data set using the IPA software focusing on the role resistin plays in carbohydrate and lipid metabolism. Twenty six of the 58 molecules involved in the network, were significantly affected by CLN (6 upregulated, 20 down-regulated).

The essential role resistin plays in the generated network is emphasized by its immediate connections to 9 other genes and molecules in the network, including D-glucose, and fatty acids, that are the building blocks of carbohydrate and lipid synthesis and storage. We identified the genes that are implicated in processes leading to obesity and diabetes according to the Ingenuity Pathways Knowledge Base. Both disorders were found to be directly connected to 10 network nodes, confirming the relevance of the generated network in these diseases. The actual number of relevant genes in the network is most likely much higher; since the Ingenuity Pathways Knowledge Base is very conservatively created containing firmly established molecular connections.

Interestingly, when we applied the software to identify genes implicated in cardiovascular diseases from the same network we found 12 network nodes, even though originally the network was generated for the role of resistin in carbohydrate and lipid metabolism. This finding underscores the interconnected nature of these diseases and the key role of resistin in their pathomechanisms. Since CLN was found to significantly affect not only resistin mRNA levels but the entire resistin-related molecular network, CLN might prove to be of major importance in the treatment of these disorders.

Thus Colostrinin may have significance in the prevention and/or treatment of obesity, as well as disorders related to and/or resulting from obesity. Colostrinin may also have significance in the control of body weight.

It will be appreciated that the invention described herein may be modified, within the scope of the claims.

1. Colostrinin for use in the treatment of obesity and/or an obesity-related disorder.

2. Colostrinin for use in the treatment for the control of body weight.

3. Colostrinin for use in the treatment of type II diabetes mellitus.

4-43. (canceled)
